(19) United States
(12) Patent Application Publication
(10) Pub. No.: US 2025/0186111 A1
(43) Pub. Date: Jun. 12, 2025

(54) PROBE, SYSTEM, AND METHOD FOR FORMING A LESION IN A TARGET TISSUE

(71) Applicant: Baylis Medical Technologies Inc., Mississauga (CA)

(72) Inventors: Ahmad Khayer Dastjerdi, Toronto (CA); Andrew Oldham, Georgetown (CA); Jimmy Huynh, York (CA); Owen Moffitt, Markham (CA); Ryan Li, Barrie (CA); Abelardo Escoto, Toronto (CA); Carolyn Holladay, York (CA); Neil Godara, Milton (CA)

(21) Appl. No.: 18/846,438

(22) PCT Filed: Mar. 13, 2023
(86) PCT No.: PCT/IB2023/052412

§ 371 Date: Sep. 12, 2024

Related U.S. Application Data
(60) Provisional application No. 63/319,894, filed on Mar. 15, 2022.

(51) Int. Cl. A61B 18/14; A61B 18/00; A61B 18/16

(52) U.S. Cl. CPC A61B 18/1492; A61B 18/16; A61B 2018/00023; A61B 2018/00083; A61B 2018/00565; A61B 2018/00577; A61B 2018/00821; A61B 2018/162

(57) ABSTRACT

A probe for forming a lesion in a target tissue includes an elongate member extending longitudinally between a proximal end and a distal end. The elongate member has an active electrode proximate the distal end for delivering ablative energy to the target tissue, and an electrically insulated section proximal of the active electrode. The active electrode includes a cooled section that is coolable by receipt of a cooling fluid delivered through the elongate member. At least a first temperature sensor is positioned external of the elongate member to measure a temperature in a heat affected zone of the target tissue. The first temperature sensor is positioned proximally of the distal end and is spaced from the cooled section.

### CROSS-REFERENCES TO RELATED APPLICATIONS

This document is related by subject matter to U.S. Provisional Patent Application No. 63/077,952 filed on Sep. 14, 2020, and International Patent Application No. PCT/IB2021/058377 filed on Sep. 14, 2021, both of which are incorporated herein by reference in their entirety.

### FIELD

This document relates medical devices. More specifically, this document relates to devices, systems, and methods for lesioning target tissue using radiofrequency energy.

### BACKGROUND OF THE ART

U.S. Pat. No. 10,610,297 (Leung et al.) discloses a device for treating spinal tissue of a patient's body including an energy source and first and second probe assemblies. Each of the probe assemblies has an electrically conductive energy delivery device electrically coupled to the energy source, and an electrothermal device for cooling the probe assembly. The device is configured so that the energy source delivers energy to the spinal tissue through the energy delivery devices in a bipolar mode that concentrates delivered energy between the energy delivery devices to create a lesion within the spinal tissue while the electrothermal devices cool the probe assemblies. Related methods of use include cooling, at times via an electrothermal device.

### SUMMARY

The following summary is intended to introduce the reader to various aspects of the detailed description, but not to define or delimit any invention.

Probes for forming lesions in target tissues are disclosed. According to some aspects, a probe for forming a lesion in a target tissue includes an elongate member extending longitudinally between a proximal end and a distal end. The elongate member has at least a first active electrode proximate the distal end for delivering ablative energy to the target tissue, and an electrically insulated section proximal of the first active electrode. The first active electrode includes a cooled section that is coolable by receipt of a cooling fluid delivered through the elongate member. At least a first temperature sensor is positioned external of the elongate member to measure a temperature in a heat affected zone of the target tissue. The first temperature sensor is positioned proximally of the distal end and is spaced from the cooled section.

In some examples, the elongate member includes at least a first fluid delivery lumen for delivering the cooling fluid through the elongate member to the cooled section. The elongate member can further include at least a first fluid return lumen for delivering the cooling fluid proximally through the elongate member. The elongate member can be configured to restrict the cooling fluid to the cooled section on delivery of the cooling fluid. For example, the probe can further include a barrier within the elongate member for restricting the cooling fluid to the cooled section on delivery of the cooling fluid. The barrier can be positioned distally of a proximal boundary of the first active electrode, to further provide the first active electrode with a non-cooled section. The first temperature sensor can be secured to the first active electrode in the non-cooled section. Alternatively, the barrier can be positioned at a proximal boundary of the first active electrode so that the cooled section makes up an entirety of the first active electrode. The first temperature sensor can be positioned proximally of the first active electrode.

In some examples, the probe includes an electrically insulative material forming the electrically insulated section or received on the electrically insulated section, and the first temperature sensor is secured to the electrically insulative material.

In some examples, the first temperature sensor is positioned to measure the temperature in a proximal projection of a thermal ablation zone.

In some examples, the first active electrode further includes a non-cooled section, and the first temperature sensor is secured to the non-cooled section.

In some examples, the first temperature sensor is spaced at least proximally from the cooled section. The temperature sensor can further be spaced radially from the cooled section.

In some examples, the elongate member includes a metallic hypotube, and the metallic hypotube includes a first section that is electrically exposed to form the first active electrode, and a second section on which an electrically insulative material is received to form the electrically insulated section. The probe can further include at least one electrical conductor for electrically connecting the first temperature sensor to a temperature control system, and the electrical conductor can be positioned between the second section of the metallic hypotube and the electrically insulative material. The metallic hypotube and the electrically insulative material can be permanently secured together, or the metallic hypotube can be removably received in the electrically insulative material.

In some examples, the probe further includes at least one electrical conductor for electrically connecting the first temperature sensor to a temperature control system, and the electrical conductor extends through a lumen of the elongate member.

In some examples, the electrically insulated section includes a hypotube fabricated from an electrically insulative material, and the first active electrode includes a metallic member secured to the electrically insulative material.

In some examples, the first temperature sensor is further configured to deliver ablative energy to the target tissue.

In some examples, the first temperature sensor includes a thermocouple junction.

Systems for forming lesions in a target tissue are also disclosed. According to some aspects, a system for forming a lesion in a target tissue includes a probe. The probe includes an elongate member and a first temperature sensor. The elongate member extends longitudinally between a proximal end and a distal end, and has at least a first active electrode proximate the distal end for delivering ablative energy to the target tissue, and an electrically insulated section proximal of the first active electrode. The first active electrode includes a cooled section that is coolable by receipt of a cooling fluid delivered through the elongate member. The first temperature sensor is positioned external of the elongate member to measure a temperature in a heat affected zone of the target tissue. The first temperature sensor is positioned proximally of the distal end and is spaced from the cooled section. The system further includes a return electrode spaced from the first active electrode, a radiofrequency generator electrically connected to the first active electrode for delivering the ablative energy to the first active electrode and electrically connected to the return electrode for returning a current to the radiofrequency generator, and a cooling fluid circulation system for delivering the cooling fluid to the probe.

Methods for forming lesions in a target tissue are also disclosed. According to some aspects, a method for forming a lesion in a target tissue includes positioning at least a first active electrode of the probe at the target tissue; delivering ablative energy from the first active electrode to form the lesion in the target tissue, while delivering a cooling fluid to a cooled section of the first active electrode; and using a first temperature sensor of the probe to monitor a temperature in a heat affected zone of the target tissue at a position proximal of a distal end of the probe and spaced from the cooled section.

In some examples, delivering the cooling fluid to the cooled section of the first active electrode includes delivering the cooling fluid to an entirety of the first active electrode.

In some examples, delivering the cooling fluid to the cooled section of the first active electrode includes, on delivery of the cooling fluid, restricting the cooling fluid to the cooled section.

In some examples, using the first temperature sensor includes monitoring the temperature proximally of the first active electrode.

In some examples, ablative energy is delivered to form a lesion having a proximal projection, and the first temperature sensor monitors the temperature in the proximal projection.

In some examples, restricting the cooling fluid to the cooled section includes using a barrier positioned at a proximal boundary of the first active electrode to restrict the cooling fluid to the first active electrode.

In some examples, restricting the cooling fluid to the cooled section includes using a barrier positioned distally of a proximal boundary of the first active electrode, to further provide the first active electrode with a non-cooled section. Using the first temperature sensor can further include monitoring the temperature adjacent the non-cooled section.

In some examples, the position is radially spaced from the cooled section.

In some examples, the method further includes delivering the ablative energy to the target tissue from the first temperature sensor.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a perspective view of an example system for forming a lesion in a target tissue, including a probe, a grounding pad, a radiofrequency generator, and a cooling fluid circulation system;

FIG. 2A is a cross section taken through the distal portion of the probe of FIG. 1, along line 2-2;

FIG. 2B is a schematic view showing the probe of FIG. 2A in use, positioned against a bone and generally perpendicular to the bone;

FIG. 2C is a schematic view showing the probe of FIG. 2A in use, positioned against a bone and generally parallel to the bone;

FIG. 3 is a cross section taken through the distal portion of another example probe;

FIG. 4 is a cross section taken through the distal portion of another example probe;

FIG. 5A is a partial plan view of another example probe;

FIG. 5B is a schematic view showing the probe of FIG. 5A in use, positioned against a bone and generally parallel to the bone;

FIG. 6 is a cross section taken through the distal portion of another example probe;

FIG. 7 is a cross section taken through the distal portion of another example probe;

FIG. 8 is a cross section taken through the distal portion of another example probe;

FIGS. 9A to 9D are partial plan views of further example probes; and

FIG. 10A is a power trace obtained with the use of a control probe, and FIG. 10B is a power trace obtained with the use of a probe as described herein.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

The terms “coupled” or “connected” or “coupling” or “connecting” as used herein can have several different meanings depending in the context in which these terms are used. For example, these terms can have a mechanical, electrical or communicative connotation. For example, as used herein, these terms can indicate that two or more elements or devices are directly connected to one another or connected to one another through one or more intermediate elements or devices via an electrical element, electrical signal, or a mechanical element depending on the particular context.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof. Furthermore, the wording “at least one of X and Y” is intended to mean only X, only Y, or both X and Y.

Terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

Generally disclosed herein are probes for forming one or more lesions in a target tissue, and related systems and methods. The probes can be configured for use in radiofrequency (RF) ablation procedures. In such procedures, an active electrode of the probe is positioned at the target site (e.g. adjacent a nerve that is the source of pain, or within a bone tumor). An RF generator creates an RF signal, which travels to the active electrode of the probe. Tissue adjacent to the active electrode is heated above body temperature as a result of the resistance to the RF current. The region in which the tissue is heated, or is expected to be heated, or will ultimately be heated, is referred to herein as a heat affected zone. The heat affected zone can extend radially outwardly from the active electrode along its length, and can further extend both distally of the active electrode (i.e. the heat affected zone has a “distal projection”) and proximally of the active electrode (i.e. the heat affected zone has a “proximal projection”). At the periphery of the heat affected zone, the tissue is raised above body temperature, but is not raised sufficiently high to cause a lesion. At the core of the heat affected zone, closest to the active electrode, the tissue temperature is raised sufficiently high to cause a lesion. The region in which the lesion is formed, or is expected to be formed, or will ultimately be formed, is referred to herein as a thermal ablation zone. The thermal ablation zone forms a part of the heat affected zone, and can extend radially outwardly from the active electrode along its length, and can further extend both distally of the active electrode (i.e. the thermal ablation zone has a “distal projection”) and proximally of the active electrode (i.e. the thermal ablation zone has a “proximal projection”).

In some examples, in order to increase the size of the thermal ablation zone, the probes described herein are configured such that at least a section of the active electrode is cooled. That is, a cooling fluid is delivered through the probe and to at least a section of the active electrode. The cooling fluid acts as a heat sink, drawing heat away from that section of the active electrode, and thus lowering the temperature of nearby tissue. The temperature reduction reduces the risk of tissue cavitation and charring. By reducing the temperature of the tissue in closest proximity to the active electrode, and thereby reducing the risks associated with high temperatures, the energy output and/or procedure time can be increased. The cooling allows for a prolonged application of RF, and the distribution of elevated temperatures from the active electrode is increased, which results in an increase of the size of the thermal ablation zone.

In the probes described herein, a temperature sensor is employed to monitor the temperature in the heat affected zone, and optionally within the thermal ablation zone. Real-time monitoring of tissue temperature can allow for immediate and automatic adjustments in the amount RF energy being delivered. This, in turn, can reduce the risk of charring and cavitation of the tissue as a result of prolonged exposure to exceedingly high temperatures. Notably, in the probes described herein, the temperature sensor is positioned to measure a temperature in the heat affected zone (i.e. in use, the temperature sensor is in contact with the tissue in the heat affected zone), but is positioned proximally of the distal end of the probe and is spaced from the cooled section of the active electrode. Positioning the temperature sensor proximally of the distal end of the probe can allow for more accurate temperature measurements when the distal end of the probe is in contact with thermally non-conductive tissues, such as bone. However, when the temperature sensor is positioned proximally of the distal end of the probe with no further modifications, temperature readings can be inaccurate and may not reflect the actual tissue temperature, as the cooling of the active electrode leads to cooling of the temperature sensor, and thus temperature readings can be lower than the actual temperature of the tissue. Thus, in the probes described herein, the temperature sensor is positioned proximally of the distal end of the probe and is also spaced from the cooled section of the active electrode, in order to allow for temperature measurements of increased accuracy, particularly when the distal end of the probe is in contact with thermally non-conductive tissue. For example, the active electrode can be provided with a non-cooled section, and the temperature sensor can be secured to the non-cooled section. In a further example, the temperature sensor can be secured to a section of the probe other than active electrode—e.g. to electrically insulative material that is proximal of the electrode—while remaining close enough to the active electrode to measure the temperature in the thermal ablation zone (i.e. the temperature sensor can be positioned to measure the temperature in the proximal projection of the lesion). In an example, the temperature sensor can be secured to a section of the probe other than active electrode, so that it is outside of the thermal ablation zone but within the heat affected zone (e.g. the temperature sensor can be positioned to measure the temperature in the proximal projection of the heat affected zone).

The probes described herein may be used in various procedures, such as ablation of nerves causing chronic pain, in particular lateral branches in the sacroiliac joint, medial branches in lumbar, thoracic, or cervical joints, genicular nerves in a knee or femur, and obturator nerves in the hip joint. The probes described herein can also be used for ablation of cancerous or non-cancerous tumors in soft or hard tissues. Furthermore, the probes described herein can be used in procedures that require placement of the probe distal end in thermally insulative tissues such as bone, fat, tendon, etc. For instance, foramina can be one of the key bony landmarks for identifying target tissues that are invisible under X-ray. The probes described herein may allow for the tip of the probe to be placed at or inside these bony landmarks without affecting the ablation performance of the probes, as the temperature sensor is not at the probe distal end. Furthermore, the probes described herein may be used in bone tumor ablation procedures in the vertebral body, where the tumor is typically surrounded by dense thermally and electrically insulative cortical bone. In such procedures, the probes described herein may allow for the distal end of the probe to be placed against the cortical bone, as the temperature sensor is not at the distal end of the probe. Furthermore, in such procedures, the probes described herein may in some examples obviate the need for a separate temperature sensing probe to be used for spinal cord safety, as the temperature sensor of the probe may serve this purpose.

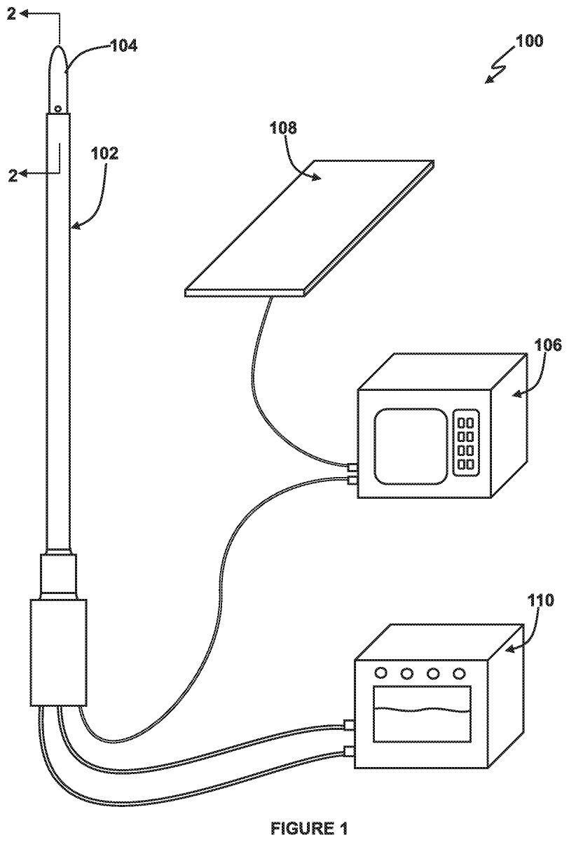

Referring now to FIG. 1, an example system 100 for forming a lesion in a target tissue is shown. The system 100 generally includes a probe 102 having an active electrode 104 (also referred to herein as a ‘first active electrode’) for delivering ablative energy to a target tissue, a radiofrequency (RF) generator 106 for delivering the ablative energy to the active electrode 104 of the probe 102, a return electrode in the form of a grounding pad 108, for returning a return current from the active electrode 104 to the radiofrequency generator 106, and a cooling fluid circulation system 110 for delivering cooling fluid to and from the probe 102. The RF generator 106 can be, for example, a generator that outputs 50 W to 100 W, that is configured for multi-channel use (i.e. more than one probe can be connected to the RF generator 106), that is usable with a cooling fluid circulation system 110, and that has a control system capable of modulating the RF energy output based on a measurable parameter of the probe 102 (e.g. temperature). The grounding pad 108 can be any suitable grounding pad usable with the RF generator 106. The cooling fluid circulation system 110 can be any cooling fluid circulation system usable with the RF generator 106 and probe 102.

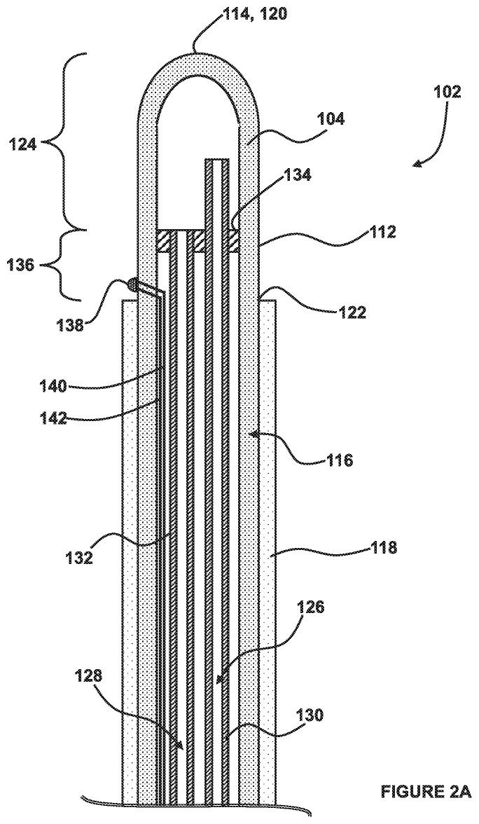

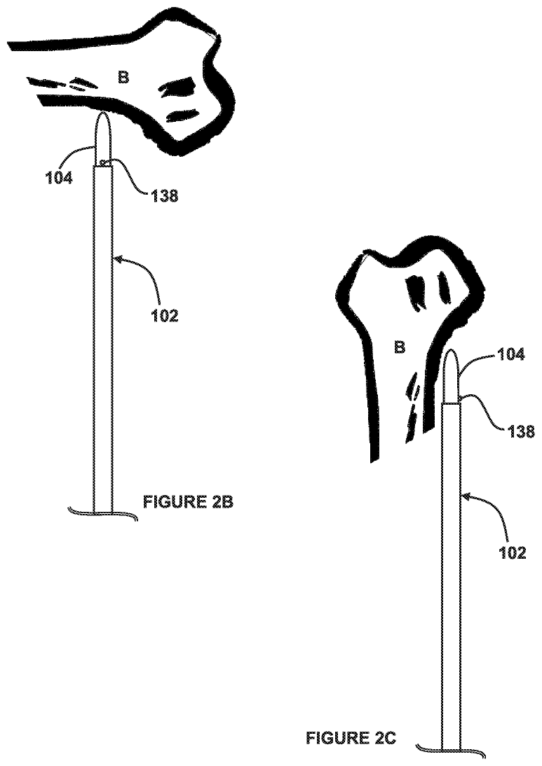

Referring now to FIG. 2A, the distal portion of the probe 102 is shown in greater detail. In the example shown, the probe 102 includes an elongate member 112, which extends longitudinally between a proximal end (not shown) and a distal end 114. As used herein, the term ‘proximal’ (and related terms such as ‘proximally’) refers to the end that is closest to the operator when the probe 102 is in use, or the direction going towards the operator when the probe 102 is in use, or the relative positioning of an element closer to the operator when the probe 102 is in use. The term ‘distal’ (and related terms such as ‘distally’) refers to the end that is furthest from the operator when the probe 102 is in use, or the direction going away from the operator when the probe 102 is in use, or the relative positioning of an element further from the operator when the probe 102 is in use. The term ‘distal end’ as used herein refers to the distalmost point of the elongate member, whereas the term ‘distal portion’ refers to the general region of the elongate member that encompasses the distal end (e.g. the term distal portion can refer to the region of the elongate member 112 that is shown in FIG. 2).

Referring still to FIG. 2A, in the example shown, the elongate member 112 is in the form of a metallic hypotube (e.g. a stainless steel hypotube) having a closed and domed distal portion. The hypotube can have a gauge of, for example, 17,18, 19, or 20.

Referring still to FIG. 2A, in the example shown, the elongate member 112 includes the active electrode 104, and an electrically insulated section 116 proximal of the active electrode 104. The active electrode 104 is proximate the distal end 114, and the electrically insulated section 116 is proximal of the active electrode 104. As used herein, the phrase ‘proximate the distal end’ indicates that the active electrode 104 is right at the distal end (as shown in FIG. 2A), or is spaced from the distal end 114 while remaining sufficiently close to the distal end 114 so that the thermal ablation zone extends distally of the distal end 114. For example, the active electrode 104 can be spaced from the distal end 114 between about 1 mm and about 40 mm.

Referring still to FIG. 2A, in the example shown, the elongate member 112 includes a section that is electrically exposed to form the active electrode 104, and an adjacent section on which an electrically insulative material 118 (e.g. polyimide) is received to form the electrically insulated section 116. In the example shown, the elongate member 112 and the electrically insulative material 118 are permanently secured together (e.g. the electrically insulative material 118 can be formed directly on the elongate member 112 as a coating); however, in alterative examples, the elongate member 112 can be removably received in the electrically insulative material 118 (e.g. the electrically insulative material 118 can be provided by an introducer through which the elongate member 112 is inserted). In yet further alternative examples (not shown), a first layer of an electrically insulative material can be permanently secured to elongate member 112, and the elongate member 112 and the first layer of electrically insulative material can be further removably received in a second layer of electrically insulative material (e.g. a second layer of electrically insulative material can be provided by an introducer through which the elongate member 112 and first layer of electrically insulative material are inserted). In yet further alternative examples (not shown), the majority of the elongate member 112 can be fabricated from an electrically insulative material, such as a sleeve of polyether ether ketone (PEEK), and the active electrode 104 can be in the form of a metallic member that is secured to the sleeve.

Referring still to FIG. 2, the active electrode has a distal boundary 120, which in the example shown is coincident with the distal end 114 of the elongate member 112, and a proximal boundary 122, which in the example shown is coincident with the end of the electrically insulative material 118. That is, in the example shown, the active electrode 104 starts where the electrically insulative material 118 ends, and extends to the distal end 114 of the elongate member 112.

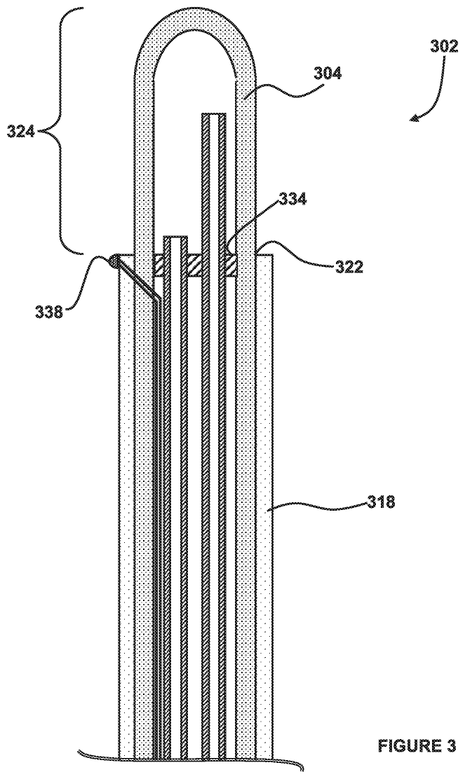

Referring still to FIG. 2A, in the example shown, the active electrode 104 includes a cooled section 124. As used herein, the term ‘cooled section’ refers to any section of an active electrode 104 that in use, is cooled by receipt of a cooling fluid (e.g. saline, a gas, or a contrast fluid). A ‘cooled section’ can be made up of only a portion of the active electrode 104 (as is the case in FIG. 2A and as will be described in further detail below), or the entirety of the active electrode 104 can make up the cooled section (as will be described below with reference to FIG. 3). A cooled section can be a section that is directly contacted by cooling fluid (as is the case in FIG. 2A), or a section that is indirectly cooled by the cooling fluid, for example via a thermal conductor between the cooled section and the cooling fluid.

Referring still to FIG. 2A, in the example shown, the probe 102 includes a fluid delivery lumen 126 for delivering cooling fluid through the elongate member 112 to the cooled section 124, and a fluid return lumen 128 for delivering the cooling fluid out of the cooled section and proximally out of the elongate member 112. In the example shown, the fluid delivery lumen 126 is provided by a first hypotube 130 and the fluid return lumen 128 is provided by a second hypotube 132. The first 130 and second 132 hypotubes can be connected to first and second fluid lines, respectively, which in turn are connected to the cooling fluid circulation system 110 (as shown in FIG. 1). The delivery of the cooling fluid through to the cooled section 124 cools the cooled section 124.

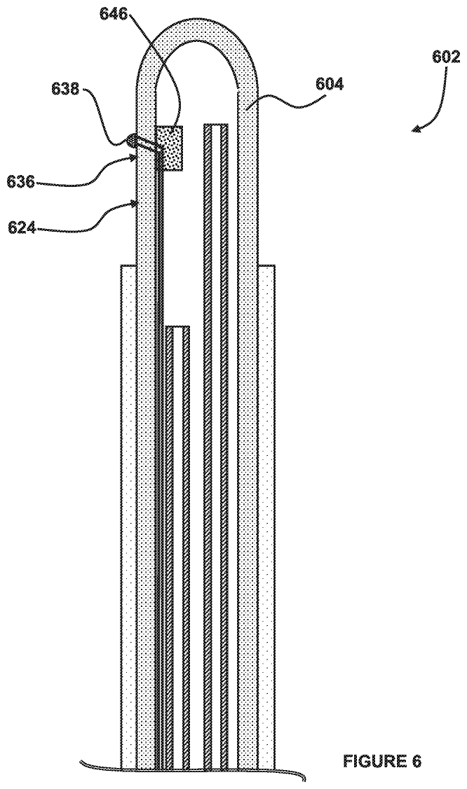

Referring still to FIG. 2A, in the example shown, the elongate member 112 is configured so that on delivery of the cooling fluid (i.e. upon the exit of the cooling fluid from the fluid delivery lumen 126 and prior to the entry of the cooling fluid into the fluid return lumen 128), the cooling fluid is restricted to the cooled section 124. That is, in the example shown, the probe 102 includes a barrier 134 within the elongate member 112 for restricting the cooling fluid to the cooled section 124 on delivery of the cooling fluid. In the example shown, the barrier 134 is positioned distally of the proximal boundary 122 of the active electrode 104, to further provide the active electrode 104 with a non-cooled section 136. As used herein the term ‘non-cooled section’ can refer to a section of the active electrode 104 that does not receive the cooling fluid (as shown in FIG. 2A), or that is separated from the cooling fluid by a thermal insulator (as shown in FIG. 6). It will be appreciated that while the temperature of the non-cooled section 136 may be affected by its proximity to the cooled section, the term “non-cooled” is used to reflect that the referenced section is configured so that it is shielded from the cooling, and is not cooled to the same extent as the cooled section.

Referring still to FIG. 2A, the probe 102 further includes a first temperature sensor 138. The first temperature 138 sensor can be, for example, a thermocouple junction, and the probe 102 can further include a pair of electrical conductors 140, 142 for electrically connecting the thermocouple junction to a temperature control system (which can be built into the RF generator). In alternative examples, the temperature sensor 138 can be another type of temperature sensor, such as thermistor, a thermometer, or an optical fluorescence sensor. In the example shown, the electrical conductors 140, 142 extend through a lumen of the elongate member 112. This can allow for bending of the probe 102 in use (e.g. to facilitate access a particular target tissue), without straining the electrical conductors 140, 142.

As shown in FIG. 2A, the first temperature sensor 138 is positioned to measure a temperature in a heat affected zone of the target tissue, and more specifically, in the thermal ablation zone. That is, the first temperature sensor 138 is not internal to the elongate member 112, but rather, is external to the elongate member 112 so that in use it is in contact with the target tissue, and is sufficiently close to the active electrode 104 to measure the temperature in the thermal ablation zone. Further, the first temperature sensor 138 is positioned proximally of the distal end 114 of the elongate member 112, and is spaced from the cooled section 124. In the example shown, the temperature sensor 138 is secured to the non-cooled section 136 of the active electrode 104 (i.e. the temperature sensor is spaced proximally from the cooled section), for example by laser welding.

As described above, the positioning of the temperature sensor 138 can allow for relatively accurate temperature readings. Particularly, referring to FIG. 2B, in instances in which the probe 102 is positioned generally perpendicular to a bone B, such that the distal end 114 is against or in close proximity to the bone B, the positioning of the temperature sensor 138 can allow for accurate measurement of the temperature in the heat affected zone, as the temperature sensor 138 is spaced from the bone B. Further, referring to FIG. 2C, in instances in which the probe 102 is positioned generally parallel to a bone B, the probe 102 can be oriented such that the temperature sensor 138 faces away from the bone B, and thus the temperature sensor 138 can allow for accurate measurement of the temperature in the heat affected zone, as the temperature sensor 138 is spaced from the bone B.

In the example shown, the temperature sensor 138 is further configured to deliver ablative energy to the target tissue. That is, the temperature sensor 138 is shorted to the ablative energy delivery circuit. In alternative examples (e.g. as shown in FIG. 3), a temperature sensor can be connected to another ablative energy source. This can facilitate the heating of tissue adjacent the temperature sensor 138, to further counteract effects of the cooling fluid on the temperature readings, which can in turn allow for further accuracy in temperature readings and/or improved control of ablative energy when the reading from the temperature sensor is used to control energy delivery. In the example shown, the temperature sensor 138 is in direct electrical contact with the active electrode 104, to short the temperature sensor 138 to the ablative energy delivery circuit.

Referring now to FIG. 3, another example of a probe is shown. In FIG. 3, features that are like those of FIG. 2A will be identified with like reference numerals as in FIG. 2A, incremented by 200.

The probe 302 of FIG. 3 is similar to the probe 102 of FIG. 2A; however, the barrier 334 is positioned at the proximal boundary 322 of the active electrode 304, so that the cooled section 324 makes up the entirety of the active electrode 304 (i.e. the active electrode 304 does not include a non-cooled section). Further, in order to be spaced from the cooled section 324, the temperature sensor 338 is secured to the electrically insulative material 318, adjacent the active electrode 304. That is, the electrically insulative material 318 thermally insulates the temperature sensor 338 from the cooled section 324. As such, in this example, the temperature sensor 338 is spaced both proximally and radially from the cooled section 324 (and thus is spaced proximally and radially from the active electrode 324 in its entirety), and the temperature sensor 338 is positioned to measure the temperature in the proximal projection of the thermal ablation zone. The temperature sensor 338 can be secured to the electrically insulative material 318 by, for example by laser welding the temperature sensor 338 to a conductive ring (not shown) that is compressed or bonded to the electrically insulative material 318.

In the example of FIG. 3, though the temperature sensor 338 is not directly secured to the active electrode 304, the temperature sensor 338 may still be configured to deliver ablative energy to the target tissue. For example, the temperature sensor 338 can be shorted to the ablative energy delivery circuit or another ablative energy source by an electrical connector.

In further alternative examples (not shown), the barrier can be omitted entirely, so that the entirety of the elongate member is cooled. In such examples, in order to be spaced from the cooled section of the active electrode, the temperature sensor can be positioned as shown in FIG. 3, so that the electrically insulative material thermally insulates the temperature sensor from the cooling fluid.

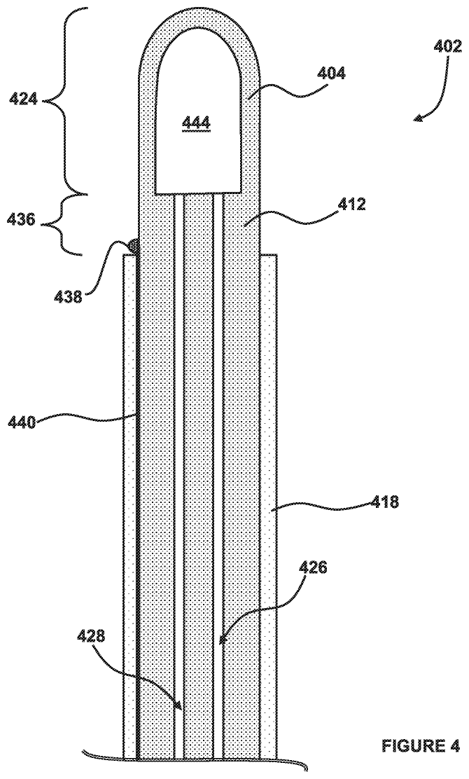

Referring now to FIG. 4, yet another example of a probe is shown. In FIG. 4, features that are like those of FIG. 2A will be identified with like reference numerals as in FIG. 2A, incremented by 300.

The probe 402 of FIG. 4 is similar to that of FIG. 2A; however, the elongate member 412 is a generally solid metallic cylinder that includes a pair of bores, which form the fluid delivery lumen 426 and the fluid return lumen 428, respectively. The elongate member 412 further includes a cooling fluid chamber 444 formed within the active electrode 404 and in fluid communication with the fluid delivery lumen 426 and the fluid return lumen 428. The cooling chamber 444 defines the cooled section 424 of the active electrode 404. In this example, on delivery of the cooling fluid, the cooling fluid is restricted to the cooling fluid chamber 444, and the section of the active electrode 404 proximal of the cooling fluid chamber 444 is a non-cooled section 436.

In the example of FIG. 4, the temperature sensor 438 is positioned similarly to that of FIG. 2A—i.e. the temperature sensor 438 is secured to the non-cooled section 436 of the active electrode 404. However, the electrical conductors (only one of which is visible—i.e. electrical conductor 440) are positioned between the elongate member 412 and the electrically insulative material 418.

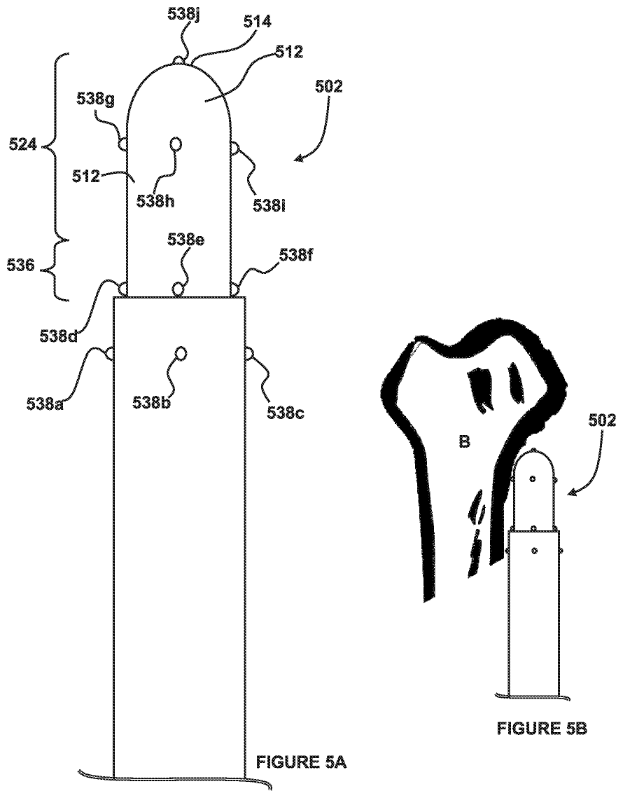

Referring now to FIG. 5A, a further alternative example of a probe is shown. In FIG. 5A, features that are like those of FIG. 2A will be identified with like reference numerals as in FIG. 2A, incremented by 400.

The probe 502 of FIG. 5A is similar to the probe of FIG. 2A; however, the probe 502 further includes additional temperature sensors. Temperature sensors 538a to 538f are positioned proximally of the distal end 514 of the elongate member 512, and are spaced from the cooled section 524. Temperature sensors 538g to 538i are positioned proximally of the distal end 514, and are in the cooled section 524. Temperature sensor 538j is at the distal end 514 and is in the cooled section 524. In this example, in use, temperature sensors 538a to 538c can serve to measure the proximal growth of the lesion, while temperature sensors 538d to 538j can measure the temperature of the tissue in contact with the active electrode 504. In this example, all of the temperature sensors 538a-j are connected to the ablative energy delivery circuit. In alternative examples, only one or only some of the temperature sensors may be connected to the ablative energy delivery circuit.

In examples in which a probe includes one or more additional temperature sensors, it may be beneficial to position the temperature sensors such that they are circumferentially spaced apart. For example, in FIG. 5A, temperature sensors 538a and 538c are spaced apart by 180 degrees; temperature sensors 538d and 538f are spaced apart by 180 degrees; and temperature sensors 538g and 538i are spaced apart by 180 degrees. Thus, referring to FIG. 5B, in instances in which the probe 502 is positioned such that temperature sensors 538a, 538d, and 538g (labelled in FIG. 5A) are against thermally non-conductive tissue, such as bone B, temperature sensors 538c, 538f, and 538i (labelled in FIG. 5A) remain spaced from the thermally non-conductive tissue, and thus are able to measure the tissue temperature accurately.

Referring now to FIG. 6, a further alternative example of a probe is shown. In

FIG. 6, features that are like those of FIG. 2A will be identified with like reference numerals as in FIG. 2A, incremented by 500.

The probe 602 of FIG. 6 is similar to the probe of FIG. 2A; however, the barrier is omitted, and the non-cooled section 636 is provided by securing a piece of thermal insulation 646 to the interior surface of the active electrode 604. The section of the active electrode 604 that is in contact with the thermal insulation 646 is thus the non-cooled section 636, and the remainder of the active electrode 604 is the cooled-section 624. The temperature sensor 638 is secured to active electrode 604 in the non-cooled section 636.

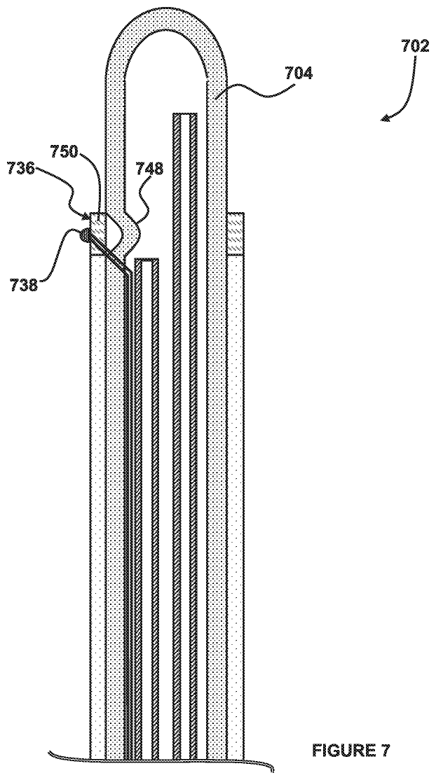

Referring now to FIG. 7, a further alternative example of a probe is shown. In FIG. 7, features that are like those of FIG. 2A will be identified with like reference numerals as in FIG. 2A, incremented by 600.

The probe 702 of FIG. 7 is similar to the probe of FIG. 2A; however, the barrier is omitted, and the non-cooled section 736 is provided by including an indent 748 in the active electrode 704, and securing a metallic ring 750 over the active electrode 704 to cover the indent 748. The metallic ring 750 forms part of the active electrode 704, but a section thereof is radially spaced from the remainder of the active electrode 704 by an air gap formed by the indent 748. The radial spacing can be, for example, between 0.2 mm and 0.6 mm. The metallic ring 750 is thus non-cooled where it covers the indent 748. That is, the portion of the metallic ring 750 that covers the indent 748 is the non-cooled section 736 of the active electrode 704, and the temperature sensor 738 is secured to this portion of the active electrode.

Referring now to FIG. 8, a further alternative example of a probe is shown. In FIG. 8, features that are like those of FIG. 2A will be identified with like reference numerals as in FIG. 2A, incremented by 700.

In the example of FIG. 8, the probe 802 includes both the active electrode 804 (also referred to in this example as a ‘distal electrode’) and a return electrode 852 (also referred to in this example as a ‘proximal electrode’). That is, the probe 802 is configured to operate in a bipolar mode, and the grounding pad 108 of FIG. 1 may be omitted. Instead, the probe 802 includes a second metallic hypotube 854, which is received on the electrically insulative material 818 and is spaced proximally from the active electrode 804. The second metallic hypotube 854 can be electrically connected to a return port of the RF generator 106 (shown in FIG. 1). The probe 802 further includes a second layer 856 of electrically insulative material, which is received on the second metallic hypotube 854, leaving a portion of the second metallic hypotube 854 exposed to form the return electrode 852.

In the example of FIG. 8, the barrier 834 is positioned so that the cooled section 824 of the active electrode 804 makes up an entirety of the distal electrode 804. Further, a region proximal of the active electrode 804 is also cooled. The temperature sensor 838 is positioned proximally of the barrier 834, and is secured to the return electrode 852.

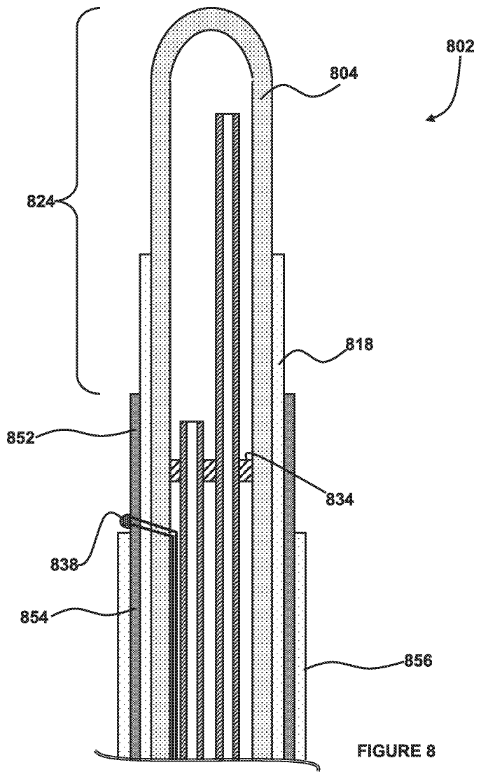

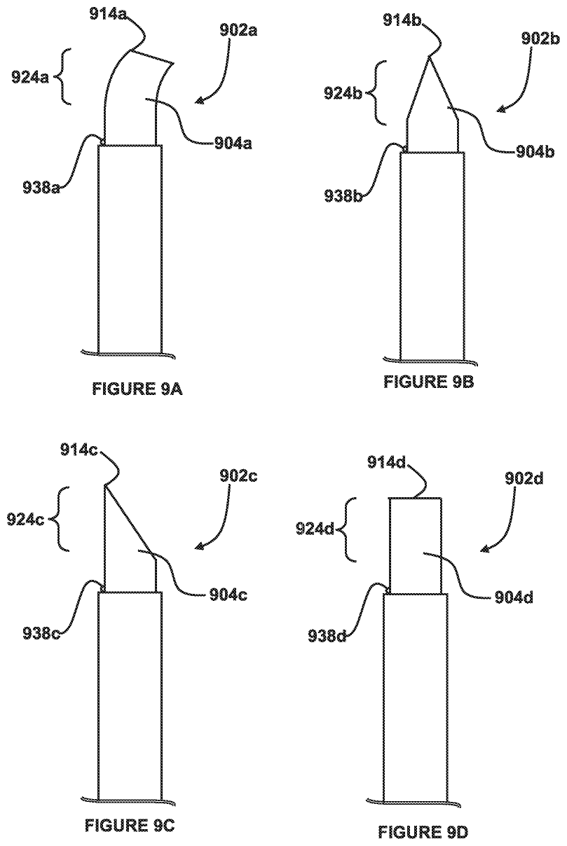

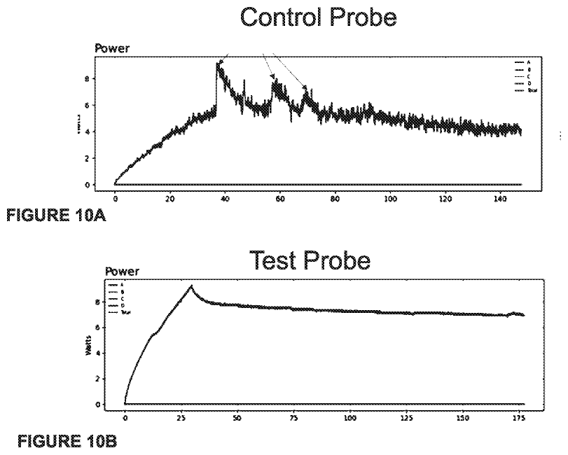

In the above examples, the active electrode is generally linear, and has a domed tip. However, as the first temperature sensor is positioned proximally of the distal end, the active electrode may take another shape, without interfering with temperature measurements or the operation of the temperature sensor. For example, as shown in FIG. 9A, the probe 902a includes an active electrode 904a that is curved, with the temperature sensor 938a positioned proximally of the distal end 914a and spaced from the cooled section 924a of the active electrode 904a. As shown in FIG. 9B, the probe 902b includes an active electrode 904b that is sharpened, with the temperature sensor 938b positioned proximally of the distal end 914b and spaced from the cooled section 924b of the active electrode 904b. As shown in FIG. 9C, the probe 902c includes an active electrode 904c that is beveled, with the temperature sensor 938c positioned proximally of the distal end 914c and spaced from the cooled section 924c of the active electrode 904c. As shown in FIG. 9D, the probe 902d includes an active electrode 904d that is square, with the temperature sensor 938d positioned proximally of the distal end 914d and spaced from the cooled section 924d of the active electrode 904d.

In the above examples, the elongate member has one fluid delivery lumen and one fluid return lumen. In alternative examples, an elongate member may include more than one fluid delivery lumen and/or more than one fluid return lumen.

In yet further alternative examples, the active electrode may not be cooled at all. In such examples, it may still be beneficial to position the temperature sensor proximally of the distal end, and spaced from the active electrode, for example to measure proximal lesion growth.

In any of the above examples, the probe may be configured to provide irrigation to the target tissue, as is described in co-pending International Patent Application No. PCT/IB2021/058377 filed on Sep. 14, 2021.

The probes described above may be used according to various methods, such as the methods described in co-pending International Patent Application No. PCT/IB2021/058377 filed on Sep. 14, 2021. Briefly, the probe may be inserted into a patient (e.g. by inserting the elongate member through an electrically insulative introducer), and the active electrode can be positioned at a target tissue. Ablative energy can then be delivered from the active electrode to form a lesion in the target tissue, while delivering a cooling fluid to the cooled section of the active electrode. The temperature sensor can be used to monitor the temperature in the heat affected zone (e.g. in the thermal ablation zone) of the target tissue, at a position proximal of the distal end of the probe and spaced from the cooled section (e.g. at a position proximal of the cooled section and/or proximal of the active electrode, at a position in a proximal projection of the thermal ablation zone, and/or at a position spaced radially from the cooled section and/or of the active electrode). Optionally, ablative energy can further be delivered from the temperature sensor to the target tissue. As described above, the cooling fluid can be delivered to the entirety of the active electrode, or to a section of the active electrode. The delivery of the cooling fluid can be restricted to the cooled section, for example using a barrier positioned at a proximal boundary of the active electrode, or using a barrier positioned distally of a proximal boundary of the electrode (to provide the active electrode with a non-cooled section).

### EXAMPLES

A probe similar to that shown in FIG. 2A (the test probe) was tested in a live porcine model. A commercially available probe, in which a temperature sensor is positioned at the distal tip of the probe, was used as a control. The probes were placed close to the lumbar medial branches, and were oriented generally perpendicular to the lamina. With the distal end of each probe pushed against the bone to ensure good contact with the probe and the lamina, ablative energy was delivered. The power was automatically adjusted by the generator in response to the temperature measured by the temperature sensor. A power delivery trace for the test probe is shown in FIG. 10B, and a power delivery trace for the control probe is shown in FIG. 10A. The power delivery traces show that the test probe results in more controlled energy delivery, with no spikes in power.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

1. A probe for forming a lesion in a target tissue, the probe comprising:
an elongate member extending longitudinally between a proximal end and a distal end, wherein the elongate member has an active electrode proximate the distal end for delivering ablative energy to the target tissue, and an electrically insulated section proximal of the active electrode, and wherein the active electrode has a cooled section that is coolable by receipt of a cooling fluid delivered through the elongate member; and
at least a first temperature sensor positioned external of the elongate member to measure a temperature in a heat affected zone of the target tissue, wherein the first temperature sensor is positioned proximally of the distal end and is spaced from the cooled section.

2. The probe of claim 1, wherein the elongate member comprises at least a first fluid delivery lumen for delivering the cooling fluid through the elongate member to the cooled section.

3. The probe of claim 2, wherein the elongate member further comprises at least a first fluid return lumen for delivering the cooling fluid proximally through the elongate member.

4. The probe of any one of claims 1 to 3, wherein the elongate member is configured to restrict the cooling fluid to the cooled section on delivery of the cooling fluid.

5. The probe of any one of claims 1 to 4, further comprising a barrier within the elongate member for restricting the cooling fluid to the cooled section on delivery of the cooling fluid, wherein the barrier is positioned distally of a proximal boundary of the active electrode, to further provide the active electrode with a non-cooled section.

6. The probe of claim 5, wherein the first temperature sensor is secured to the active electrode in the non-cooled section.

7. The probe of any one of claims 1 to 4 further comprising a barrier within the elongate member for restricting the cooling fluid to the cooled section on delivery of the cooling fluid, wherein the barrier is positioned at a proximal boundary of the active electrode so that the cooled section makes up an entirety of the active electrode.

8. The probe of any one of claims 1 to 7, wherein the first temperature sensor is positioned proximally of the active electrode.

9. The probe of any one of claims 1 to 8, wherein the probe comprises an electrically insulative material forming the electrically insulated section or received on the electrically insulated section, and wherein the first temperature sensor is secured to the electrically insulative material.

10. The probe of any one of claims 1 to 9, wherein the first temperature sensor is positioned to measure the temperature in a proximal projection of a thermal ablation zone.

11. The probe of claim 1, wherein the active electrode further comprises a non-cooled section, and wherein the first temperature sensor is secured to the non-cooled section.

12. The probe of claim 1, wherein the first temperature sensor is spaced at least proximally from the cooled section.

13. The probe of claim 12, wherein the first temperature sensor is further spaced radially from the cooled section.

14. The probe of any one of claims 1 to 8, wherein the elongate member comprises a metallic hypotube, wherein the metallic hypotube comprises a first section that is electrically exposed to form the active electrode, and a second section on which an electrically insulative material is received to form the electrically insulated section.

15. The probe of claim 14, further comprising at least one electrical conductor for electrically connecting the first temperature sensor to a temperature control system, wherein the electrical conductor is positioned between the second section of the metallic hypotube and the electrically insulative material.

16. The probe of claim 14 or 15, wherein the metallic hypotube and the electrically insulative material are permanently secured together.

17. The probe of claim 14 or 15, wherein the metallic hypotube is removably received in the electrically insulative material.

18. The probe of any one of claims 1 to 14, further comprising at least one electrical conductor for electrically connecting the first temperature sensor to a temperature control system, wherein the electrical conductor extends through a lumen of the elongate member.

19. The probe of any one of claims 1 to 8, wherein the electrically insulated section comprises a hypotube fabricated from an electrically insulative material, and the active electrode comprises a metallic member secured to the electrically insulative material.

20. The probe of any one of claims 1 to 19, wherein the first temperature sensor is further configured to deliver ablative energy to the target tissue.

21. The probe of any one of claims 1 to 20, wherein the first temperature sensor comprises a thermocouple junction.

22. A system for forming a lesion in a target tissue, comprising:
a probe comprising an elongate member and a first temperature sensor, wherein the elongate member extends longitudinally between a proximal end and a distal end, wherein the elongate member has an active electrode proximate the distal end for delivering ablative energy to the target tissue, and an electrically insulated section proximal of the active electrode, wherein the active electrode comprises a cooled section that is coolable by receipt of a cooling fluid delivered through the elongate member, wherein the first temperature sensor is positioned external of the elongate member to measure a temperature in a heat affected zone of the target tissue, and wherein the first temperature sensor is positioned proximally of the distal end and is spaced from the cooled section;
a return electrode spaced from the active electrode;
a radiofrequency generator electrically connected to the active electrode for delivering the ablative energy to the active electrode and electrically connected to the return electrode for returning a return current to the radiofrequency generator; and
a cooling fluid circulation system for delivering the cooling fluid to the probe.

23. A method for forming a lesion in a target tissue, comprising:
positioning an active electrode of a probe at the target tissue;
delivering ablative energy from the active electrode to form the lesion in the target tissue, while delivering a cooling fluid to a cooled section of the active electrode; and
using a first temperature sensor of the probe to monitor a temperature in a heat affected zone of the target tissue at a position proximal of a distal end of the probe and spaced from the cooled section.

24. The method of claim 23, wherein delivering the cooling fluid to the cooled section of the active electrode comprises delivering the cooling fluid to an entirety of the active electrode.

25. The method of claim 23, wherein delivering the cooling fluid to the cooled section of the active electrode comprises, on delivery of the cooling fluid, restricting the cooling fluid to the cooled section.

26. The method of any one of claims 23 to 25, wherein using the first temperature sensor comprises monitoring the temperature proximally of the active electrode.

27. The method of any one of claims 23 to 26, wherein the lesion has a proximal projection, and the first temperature sensor monitors the temperature in the proximal projection.

28. The method of claim 25, wherein restricting the cooling fluid to the cooled section comprising using a barrier positioned at a proximal boundary of the active electrode to restrict the cooling fluid to the active electrode.

29. The method of claim 25, wherein restricting the cooling fluid to the cooled section comprising using a barrier positioned distally of a proximal boundary of the active electrode, to further provide the active electrode with a non-cooled section.

30. The method of claim 29, wherein using the first temperature sensor comprises monitoring the temperature adjacent the non-cooled section.

31. The method of any one of claims 23 to 30, wherein the position is radially spaced from the cooled section.

32. The method of any one of claims 21 to 29, further comprising delivering the ablative energy to the target tissue from the first temperature sensor.
